(19) United States
(12) Patent Application Publication
(10) Pub. No.: US 2023/0078511 A1
(43) Pub. Date: Mar. 16, 2023

(54) METHODS OF USING MOMELOTINIB TO TREAT JOINT INFLAMMATION

(71) Applicant: Sierra Oncology, Inc., San Mateo, CA (US)

(72) Inventors: Igor THEURL, San Mateo, CA (US); Ryan James HANSEN, San Mateo, CA (US); Christian Andrew HASSIG, San Mateo, CA (US)

(21) Appl. No.: 17/759,633

(22) PCT Filed: Jan. 28, 2021
(86) PCT No.: PCT/US2021/015383

§ 371 Date: Jul. 28, 2022

Related U.S. Application Data
(60) Provisional application No. 62/967,376, filed on Jan. 29, 2020.
(60) Provisional application No. 63/033,082, filed on Jun. 1, 2020.

(51) Int. Cl. A61K 31/5377; A61K 45/06; A61P 19/02

(52) U.S. Cl. CPC A61K 31/5377; A61K 45/06; A61P 19/02

(57) ABSTRACT

The disclosure provides methods of treating joint inflammation, including rheumatoid arthritis, in a subject using mo-melotinib (MMB).

### 1. CROSS-REFERENCE TO RELATED APPLICATIONS

This application claims priority to U.S. Provisional Application No. 62/967,376, filed on Jan. 29, 2020; and U.S. Provisional Application No. 63/033,082, filed on Jun. 1, 2020, both of which are hereby incorporated by reference in their entirety.

### 2. BACKGROUND

Janus kinases (JAKs) serve as signaling hubs orchestrating inflammation, innate and adaptive immunity and erythropoiesis. As such, JAK inhibitors (JAKi) have been approved across several indications including joint inflammation, with others in late stage clinical development. Unfortunately, some of these agents cause suppression of JAK-dependent erythropoiesis, thereby exacerbating inflammation-associated anemia, leading to potential under-dosing and reduced therapeutic benefit.

For example, approved oral therapies for rheumatoid arthritis, including the JAK inhibitor class, are associated with a range of side effects including myelosuppression and anemia, which may be exacerbated under conditions of anemia of chronic disease (ACD). ACD, sometimes referred to anemia of inflammation, can result in reduced circulating iron leading to an iron-restricted inhibition of erythropoiesis. Up to 65% of rheumatoid arthritis patients may also experience ACD. Currently these side-effects are managed by label driven dose modifications, which can lead to suboptimal exposure and potential reduced clinical benefit.

Clinically, ACD can be differentiated from other forms of anemia. (Madu, Med Princ Pract. 2017;26(1):1-9.) The major difference between ACD and iron-deficiency anemia (IDA) is that in IDA there is an absolute lack (serum ferritin below 30 ng/mL) of iron, (Poggiali, Eur J Intern Med. 2014;25:12-17.) while the pathogenesis of ACD is multifactorial with iron sequestered so that it cannot be utilized in erythropoiesis. In ACD, transferrin is increased while serum iron and transferrin saturation are reduced, while the erythrocyte-free protoporphyrin, serum ferritin, and marrow-stainable iron are increased. (Spivak, Oncology 2002;16:25-33.) Hepcidin is also increased.(Ganz, N Engl J Med. 2019;381:1148-1157.) These biomarkers may help identify rheumatoid arthritis patients who also have iron-restricted anemia of chronic disease that may receive limited clinical benefit from currently approved JAK inhibitors.

Still’s disease (SD) is an autoinflammatory disease characterized by spiking fever, rash, polyarthralgia, sore throat and even life-threatening complications, such as macrophage activation syndrome and fulminant hepatitis. Excessive and inappropriate production of cytokines is a cornerstone in SD pathogenesis. Serum ferritin is considered a useful diagnostic and disease activity marker for Still’s disease. (Hu, Annals of the Rheumatic Diseases 2020;79:842-844.) The serum ferritin levels are usually higher than in any other autoimmune or inflammatory disease and very high levels between 3,000-30,000 µg/L are not uncommon. (Bagnari, Rheumatol Int. 2010;30(7):855-62.) A cutoff for ferritin levels of 1,000 µg/L has been used to indicate Still’s disease in many studies.( Fautrel, Joint Bone Spine 2002;69(4):355-7.) Hepcidin may also be increased in this disease. (Lopez-Aparicio, EJCRIM 2015;2(6).) JAK inhibitors which block the proinflammatory effect of a wide range of cytokines, could be beneficial in SD patients. A JAK inhibitor able to block the proinflammatory effect of a wide range of cytokines and also able decrease hepcidin via ACVR1 inhibition could be more beneficial in this disease.

The JAKi momelotinib (MMB) has been shown to correct anemia in a rat model, an effect that has been clinically reproduced in myelofibrosis patients treated with MMB. Subsequently, the molecular basis for MMB’s anemia benefit was determined to be a consequence of its potent inhibition of Activin Receptor Type 1 (ACVR1), resulting in decreased hepcidin and, as a consequence, increased systemic iron availability and improved erythropoiesis.

Methods for treating and preventing joint inflammation, including rheumatoid arthritis, concurrently with treating and preventing inflammation-associated anemia are of interest.

Methods for treating and preventing rheumatoid arthritis, concurrently with treating and preventing inflammation-associated anemia are of interest.

Methods for treating and preventing Still’s Disease, concurrently with treating and preventing inflammation-associated anemia are of interest.

### 3. SUMMARY OF THE INVENTION

Disclosed herein are methods of treating joint inflammation comprising administering to a subject in need thereof a therapeutically effective amount of momelotinib (MMB) or a pharmaceutically acceptable salt thereof. In some embodiments the subject has arthritis. In some embodiments the subject has rheumatoid arthritis.

In some embodiments, ameliorating one or more symptoms of arthritis comprises a reduction in joint volume. In some embodiments, the reduction in joint volume is at least 2% compared to the joint volume prior to administering the therapeutically effective amount of MMB. In some embodiments, the reduction in joint volume is at least 5% compared to the joint volume prior to administering the therapeutically effective amount of MMB.

In some embodiments, treating joint inflammation comprises ameliorating swelling of affected tissue, as measured by a reduction in diameter of an inflamed joint. In some embodiments, the reduction in diameter of the inflamed joint is at least 2% compared to the diameter of the inflamed joint prior to administering the therapeutically effective amount of MMB.

In some embodiments, treating joint inflammation comprises reducing blood neutrophil count in the subject following administering the therapeutically effective amount of MMB. In some embodiments, treating joint inflammation comprises reducing myeloid cell infiltration in spleen and/or synovial tissue. In some embodiments, treating joint inflammation comprises reducing in synovial tissue at least one of granulocytes, macrophages, monocytes and neutrophils. In some embodiments, the inflammatory macrophages are CD11b⁺.

In some embodiments, the reducing in synovial tissue the amount of at least one of granulocytes or macrophages is compared to the amount of at least one of granulocytes or macrophages in synovial tissue from the subject prior to administering MMB. In some embodiments, the amount of at least one of granulocytes or macrophages is reduced by at least 10%. In some embodiments, the amount of at least one of granulocytes or macrophages is reduced by at least 15%, at least 20%, at least 30%, at least 40%, at least 50%, or at least 60%.

In some embodiments, administering the therapeutically effective amount of MMB or a pharmaceutically acceptable salt thereof results in reduction in the number of IL-17A-producing helper T lymphocytes (Th17 cells) in the spleen of the subject compared to the number of Th17 cells in the spleen of the subject prior to administering MMB or a pharmaceutically acceptable salt thereof.

In some embodiments, administering the therapeutically effective amount of MMB or a pharmaceutically acceptable salt thereof results in reduction in the number of CD4⁺ and/or CD8⁺ T cells in synovial tissue of an inflamed joint of the subject compared to the number CD4⁺ and/or CD8⁺ T cells in synovial tissue of the inflamed joint prior to administering MMB or a pharmaceutically acceptable salt thereof.

In some embodiments, the MMB or a pharmaceutically acceptable salt thereof is administered orally. In some embodiments, the MMB or a pharmaceutically acceptable salt thereof is administered daily or weekly. In some embodiments, the MMB or a pharmaceutically acceptable salt thereof is administered intermittently.

In some embodiments, the therapeutically effective amount of MMB or a pharmaceutically acceptable salt thereof is 25-500 mg/day. In some embodiments, the therapeutically effective amount is selected from: 25 mg/day, 50 mg/day, 100 mg/day, 200 mg/day, 300 mg/day, 400 mg/day, and 500 mg/day. In some embodiments, the therapeutically effective amount is 200 mg/day.

In some embodiments, the therapeutically effective amount of MMB or a pharmaceutically acceptable salt thereof is administered for a period of 1 week or more. In some embodiments, the therapeutically effective amount is administered for a period of 2 weeks or more or 3 weeks or more. In some embodiments, the therapeutically effective amount is administered for a period of 3 weeks or more.

In some embodiments of the disclosure, the subject is a mammal. In some embodiments, the subject is human.

Also disclosed herein are methods of treating joint inflammation in a subject comprising administering to a subject in need thereof a therapeutically effective amount of momelotinib (MMB) or a pharmaceutically acceptable salt thereof, and administering to the subject one or more additional anti-inflammatory agents. In some embodiments, the one or more additional anti-inflammatory agents are anti-arthritic agents.

### 4. BRIEF DESCRIPTION OF THE SEVERAL VIEWS OF THE DRAWINGS

These and other features, aspects, and advantages of the present invention will become better understood with regard to the following description, and accompanying drawings, where:

FIG. 1A shows a schematic of JAK and ACVR1 signaling; FIG. 1B shows a schematic noting downstream effectors of signaling via JAKs and ACVR1.

FIG. 2A is a photograph of the hind limbs of rats treated with and without MMB;

FIG. 2B is a graphical representation of the diameters of hind limbs in rats treated with vehicle or various concentrations of MMB, as measured with a caliper (sum for both hind limbs presented); FIG. 2C are representative photos of H&E-stained sections of hind limb ankle joints from non-immunized and PG-PS-immunized rats treated with vehicle or MMB.

FIG. 3A shows changes in number of granulocytes in PG-PS rats following treatment with various concentrations of MMB as a percentage of CD45⁺ spleen leukocytes; FIG. 3B shows changes in the number of synovial granulocytes, CD11b⁺ macrophages, and monocytes in samples isolated from hind limb ankle joint pairs of rats treated with and without MMB.

FIG. 4A shows changes in the number of splenic Th1, Th17 and Treg cells in PG-PS rats following treatment with MMB as a percentage of CD45⁺ spleen T cells; FIG. 4B shows changes in the number of synovial CD4⁺ and CD8⁺ T cells in hind limb ankle joints of rats following treatment with MMB.

FIG. 5 is a schematic outlining the experimental process for arthritis induction in mice by injection of collagen antibody.

FIGS. 6A-6C show changes in mean total arthritis score (FIG. 6A), mean rear paw arthritis score (FIG. 6B), and mean rear paw thickness (FIG. 6C) in CAIA mice treated with vehicle, dexamethasone, etanercept or MMB (20, 30, or 50 mg/kg).

FIGS. 7A-7F shows MMB reduces systemic inflammation, joint damage and Th17 cell differentiation in the PG-PS rat RA. Shown are a heat presenting spleen and liver cytokine expression, blood leukocyte, and granulocyte counts (FIG. 7A), representative photos and sum hind limb thickness indicating MMB treatment reduces hind limb joint swelling (FIG. 7B), representative H&E-stained hind limb joint sections indicating MMB therapy alleviates cartilage damage (FIG. 7C), flow cytometry data depicting MMB reduces infiltration of neutrophils and macrophages in hind limb synovia of animals after 21 days of treatment (FIG. 7D), flow cytometry data depicting MMB inhibits differentiation of splenic Th17 cells after 21 days of treatment without affecting Th1 and regulatory Treg cells (FIG. 7E), and flow cytometry data depicting MMB significantly reduces CD4⁺ and CD8⁺ T cell infiltration in hind-limb synovial tissue after 21 days of treatment (FIG. 7F).

FIG. 8 shows MMB decrease hepcidin and corrects anemia in the PG-PS rat RA.

FIGS. 9A-9C shows clinical activity and non-inferiority of MMB in the CAIA murine arthritis. Shown are mean clinical scoring of animals (FIG. 9A), mean rear paw scoring (FIG. 9B), and mean rear paw thickness (FIG. 9C).

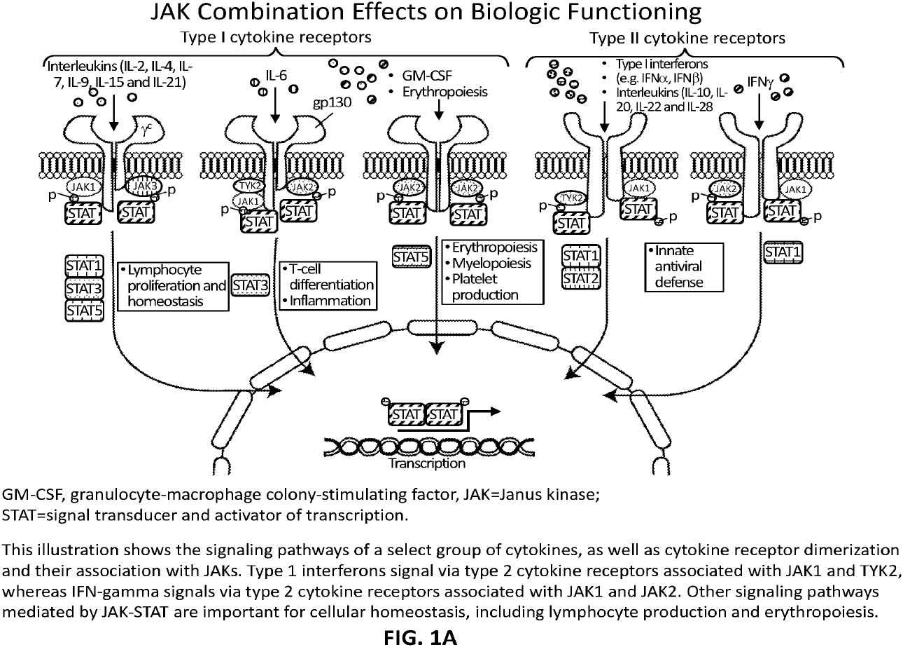

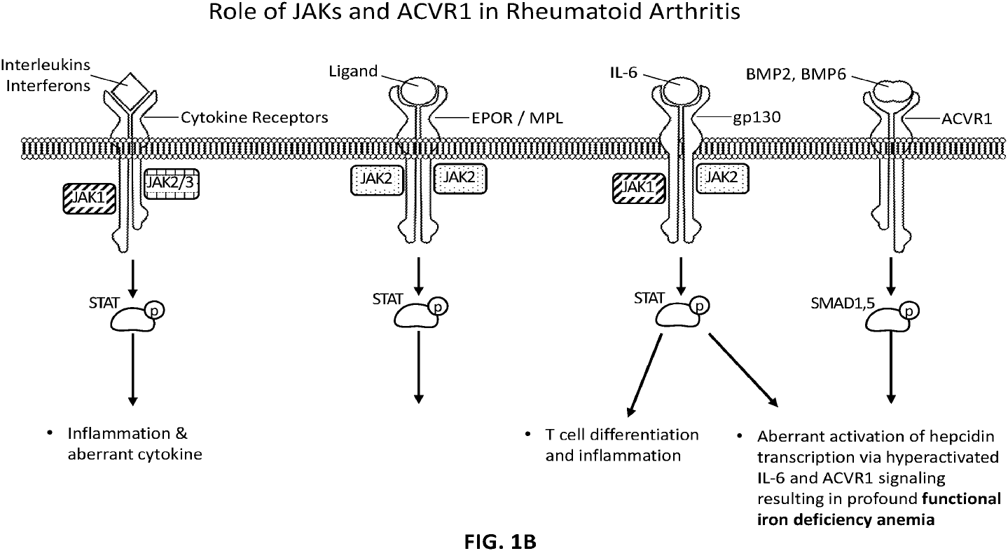

### 5. DETAILED DESCRIPTION OF THE INVENTION

### Definitions

Terms used in the claims and specification are defined as set forth below unless otherwise specified.

The term “ameliorating” refers to any therapeutically beneficial result in the treatment of a disease state, e.g., an arthritic disease state, including prophylaxis, lessening in the severity or progression, remission, or cure thereof.

The term “mammal” as used herein includes both humans and non-humans and include but is not limited to humans, non-human primates, canines, felines, murines, bovines, equines, and porcines.

The term percent “identity” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

For purposes herein, percent identity and sequence similarity is performed using the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

As used herein, the term “subject” broadly refers to any animal, including but not limited to, human and non-human animals (e.g., dogs, cats, cows, horses, sheep, pigs, poultry, fish, crustaceans, etc.).

As used herein, the term “effective amount” refers to the amount of a composition (e.g., a synthetic peptide) sufficient to effect beneficial or desired results. An effective amount can be administered in one or more administrations, applications or dosages and is not intended to be limited to a particular formulation or administration route.

The term “therapeutically effective amount” is an amount that is effective to ameliorate a symptom of a disease. A therapeutically effective amount can be a “prophylactically effective amount” as prophylaxis can be considered therapy.

As used herein, the terms “administration” and “administering” refer to the act of giving a drug, prodrug, or other agent, or therapeutic treatment (e.g., peptide) to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal or lingual), ear, rectal, vaginal, by injection (e.g., intravenously, subcutaneously, intratumorally, intraperitoneally, etc.) and the like.

As used herein, the term “treatment” means an approach to obtaining a beneficial or intended clinical result. The beneficial or intended clinical result can include alleviation of symptoms, a reduction in the severity of the disease, inhibiting an underlying cause of a disease or condition, steadying diseases in a non-advanced state, delaying the progress of a disease, and/or improvement or alleviation of disease conditions.

As used herein, the term “pharmaceutical composition” refers to the combination of an active ingredient with a carrier, inert or active, making the composition especially suitable for therapeutic or diagnostic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

It must be noted that, as used in the specification and the appended claims, the singular forms “a,” “an” and “the” include plural referents unless the context clearly dictates otherwise.

### Momelotinib (MMB)

In one aspect, the present disclosure provides for methods of use of the compound momelotinib (MMB). MMB is sometimes referred to as CYT387. The compound MMB is also identified by the chemical name: N-(cyanomethyl)-4-(2-(4-morpholinophenylamino)pyrimidin-4-yl)benzamide. Salts, including pharmaceutically acceptable salts, solvates, hydrates and/or polymorph forms of MMB can find use in the subject methods disclosed herein.

MMB is a compound that is disclosed in international patent application no. PCT/US2015/035316 and international patent publication no. WO2008/109943, the disclosures of which are herein incorporated by reference. The skilled artisan will find methods that can be used to synthesize MMB in international patent publication no. WO2008/109943.

Table 1 shows the MMB compound structure.

Table 1

MMB Structure

| | |
|---|---|
| Description | Structure |
| MMB structure | 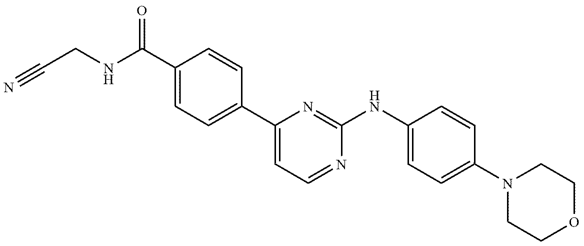 [SMILES: N#CCNC(=O)c1ccc(-c2ccnc(Nc3ccc(N4CCOCC4)cc3)n2)cc1] |

In some cases, a pharmaceutically acceptable salt of MMB is utilized. In some cases, the MMB salt that finds use in the subject methods is a hydrochloride salt. In certain cases, the MMB salt is a dihydrochloride salt. In some cases, the MMB salt is a monohydrochloride salt. In some cases, the MMB salt is a hydrate, such as a monohydrate.

A “solvate” is formed by the interaction of a solvent and a compound. Solvates of salts of the compound described herein are also provided. When the solvent is water, the solvate may be referred to as a hydrate. Hydrates of MMB or MMB salts can also find use in the subject methods.

In some embodiments, the MMB salt is MMB dihydrochloride monohydrate.

In some embodiments, the MMB salt is MMB dihydrochloride anhydrous.

In some embodiments, the MMB or MMB salt composition that is administered is present in a polymorph form, such as a polymorph form that is described in U.S. Pat. No. 9,469,613, the disclosure of which is herein incorporated by reference.

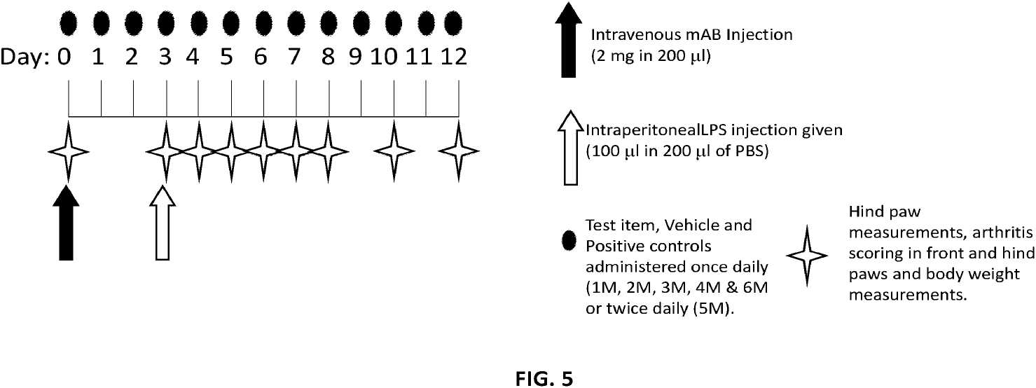

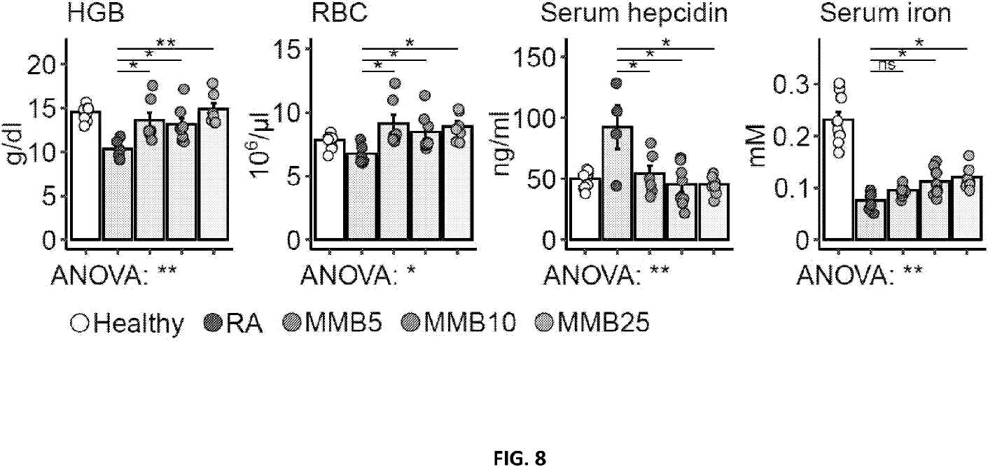

In certain instances, the MMB polymorph form is MMB dihydrochloride monohydrate Form II. The crystalline form of the MMB dihydrochloride monohydrate Form II can have crystals having unit cell parameters at T=100° K of: a =10.2837(6) Å, b=10.4981(6) Å,c=11.5143(7) Å,α=83.297(2)°, β=87.649(2)°, y=67.445(2)°, and a triclinic P-1 space group. The crystalline form of the MMB dihydrochloride monohydrate Form II can be characterized by an x-ray powder diffraction (XRPD) pattern substantially as set forth in FIG. 5 of U.S. Pat. No. 9,469,613. The crystalline form of the MMB dihydrochloride monohydrate Form II can be characterized by an x-ray powder diffraction (XRPD) pattern having peaks at about 7.7°, 19.3°, 24.0°, 25.7°, and 29.6°2-θ±0.2° 2-θ. The crystalline form of the MMB dihydrochloride monohydrate Form II can be characterized by differential scanning calorimetry (DSC) pattern substantially as set forth in FIG. 8 of U.S. Pat. No. 9,469,613. The crystalline form of the MMB dihydrochloride monohydrate Form II can be characterized by a dynamic vapor sorption (DVS) pattern substantially as set forth in FIG. 14 of U.S. Pat. No. 9,469,613.

In certain instances, the MMB of the present disclosure may have from 1 to n hydrogen atoms replaced by a deuterium atom (D), in which n is the number of hydrogen atoms in the compound. Such deuterated MMB compounds may increase resistance to metabolism and thus may be useful for increasing the half-life of the compounds described herein when administered to a mammal. See, e.g., Foster, “Deuterium Isotope Effects in Studies of Drug Metabolism,” Trends Pharmacol. Sci., 5(12):524-527 (1984).

### Rheumatoid Arthritis With Anemia of Chronic Disease (ACD)

Rheumatoid arthritis patients or Still’s Disease patients that also suffer from iron-restricted anemia of chronic disease may show limited clinical benefit from currently approved JAK inhibitors. These patients can be identified by measuring certain biomarkers. In some embodiments these patients may have transferrin levels that are above the normal range. In some embodiments these patients may also have serum iron and transferrin saturation that are below the normal ranges. In some embodiments these patients may also have hepcidin levels above the normal range.

### Administration

As disclosed herein, the methods of the invention include administration of an effective amount of MMB. In an embodiment, the effective amount of MMB is administered as a monotherapy. The present disclosure provides for a method of treatment wherein the effective amount of MMB is administered to a subject. The term “effective amount” or “therapeutically effective amount” refers to an amount that is effective to ameliorate a symptom of a disease, e.g. as described herein.

In the treatment or prevention of diseases and conditions described herein an appropriate dosage level of MMB will generally be about 0.01 to 500 mg per kg patient body weight per day which can be administered in single or multiple doses. In some cases, the dosage level will be about 0.1 to about 250 mg/kg per day; such as about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient. The dosage may be selected, for example to any dose within any of these ranges, for therapeutic efficacy and/or symptomatic adjustment of the dosage to the patient to be treated. The MMB can be administered on a regimen of 1 to 4 times per day, preferably once or twice per day.

It will be understood that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Also disclosed herein, the methods of this disclosure include a combination therapy administering an effective amount of MMB and co-administering a second effective amount of a further treatment. Further treatments include, but are not limited to, administering any convenient additional agent that finds use in treating a disease or conditions associated with CKD. In some instances, the additional agent is an antihypertensive agent, an antilipemic agent, or an antidiabetic agent.

Coadministered encompasses methods where MMB and the further treatment are given simultaneously, where MMB and the further treatment are given sequentially, and where either one of, or both of, MMB and the further treatment are given intermittently or continuously, or any combination of: simultaneously, sequentially, intermittently and/or continuously. The skilled artisan will recognize that intermittent administration is not necessarily the same as sequential because intermittent also includes a first administration of an agent and then another administration later in time of that very same agent. Moreover, the skilled artisan understands that intermittent administration also encompasses sequential administration in some aspects because intermittent administration does include interruption of the first administration of an agent with an administration of a different agent before the first agent is administered again. Further, the skilled artisan will also know that continuous administration can be accomplished by a number of routes including i.v. drip or feeding tubes, etc.

Furthermore, and in a more general way, the term “coadministered” encompasses any and all methods where the individual administration of MMB and the individual administration of the further treatment to a subject overlap during any timeframe.

In one aspect, the frequency of administration of MMB or the further treatment to a subject includes, but is not limited to, Q1d, Q2d, Q3d, Q4d, Q5d, Q6d, Q7d, Q8d, Q9d, Q10d, Q14d, Q21d, Q28d, Q30d, Q90d, Q120d, Q240d, or Q365d. The term “QnD or qnd” refers to drug administration once every “n” days. For example, QD (or qd) refers to once every day or once daily dosing, Q2D (or q2d) refers to a dosing once every two days, Q7D refers to a dosing once every 7 days or once a week, Q5D refers to dosing once every 5 days, and so on. In one aspect, MMB and the further treatment are administered on different schedules.

In some aspects, the present disclosure provides for methods where either one of, or both of, or any combination thereof, MMB and/or a further treatment are administered by a route selected from the group consisting of: intravenous, subcutaneous, cutaneous, oral, intramuscular, and intraperitoneal. In some aspects, the present disclosure provides for methods where either one of, or both of, or any combination thereof, MMB and/or a further treatment are administered intravenously. In some aspects, the present disclosure provides for methods where either one of, or both of, or any combination thereof, MMB and/or a further treatment are administered orally.

It is understood by the skilled artisan that the unit dose forms of the present disclosure may be administered in the same or different physicals forms, i.e. orally via capsules or tablets and/or by liquid via i.v. infusion, and so on. Moreover, the unit dose forms for each administration may differ by the particular route of administration. Several various dosage forms may exist for either one of, or both of, MMB and a further treatment. Because different medical conditions can warrant different routes of administration, the same components of a combination of MMB and a further treatment described herein may be exactly alike in composition and physical form and yet may need to be given in differing ways and perhaps at differing times to alleviate the condition. For example, a condition such as persistent nausea, especially with vomiting, can make it difficult to use an oral dosage form, and in such a case, it may be necessary to administer another unit dose form, perhaps even one identical to other dosage forms used previously or afterward, with an inhalation, buccal, sublingual, or suppository route instead or as well. The specific dosage form may be a requirement for certain combinations of MMB and a further treatment, as there may be issues with various factors like chemical stability or pharmacokinetics.

In some aspects, the effective amount of MMB is less than or equal to the maximum tolerated dose (MTD), less than or equal to the highest non-severely toxic dose (HNSTD), or less than or equal to the No-observed-adverse-effect-level (NOAEL).

In general, the compounds of the present disclosure will be administered in a therapeutically effective amount by any of the accepted modes of administration for agents that serve similar utilities. the actual amount of the compound of the present technology, i.e., the active ingredient, will depend upon numerous factors such as the severity of the disease to be treated, the age and relative health of the subject, the potency of the compound used, the route and form of administration, and other factors well known to the skilled artisan. The drug can be administered at least once a day, preferably once or twice a day.

An effective amount of such agents can readily be determined by routine experimentation, as can the most effective and convenient route of administration and the most appropriate formulation. Various formulations and drug delivery systems are available in the art. See, e.g., Gennaro, A.R., ed. (1995) Remington’s Pharmaceutical Sciences, 18th ed., Mack Publishing Co.

A therapeutically effective dose can be estimated initially using a variety of techniques well-known in the art. Initial doses used in animal studies may be based on effective concentrations established in cell culture assays. Dosage ranges appropriate for human subjects can be determined, for example, using data obtained from animal studies and cell culture assays.

An effective amount or a therapeutically effective amount or dose of an agent, e.g., MMB, refers to that amount of the agent or compound that results in amelioration of symptoms or a prolongation of survival in a subject. Toxicity and therapeutic efficacy of such molecules can be determined by standard pharmaceutical procedures in cell cultures or experimental animals, e.g., by determining the maximum tolerated dose (MTD), the highest non-severely toxic dose (HNSTD), the No-observed-adverse-effect-level (NOAEL), or the LD₅₀ (the dose lethal to 50% of the population) and the ED₅₀ (the dose therapeutically effective in 50% of the population). The dose ratio of toxic to therapeutic effects is therapeutic index, which can be expressed as the ratio of the MTD, HNSTD, NOAEL, or LD₅₀ to the ED₅₀. Agents that exhibit high therapeutic indices are preferred.

The effective amount or therapeutically effective amount is the amount of the compound or pharmaceutical composition that will elicit the biological or medical response of a tissue, system, animal or human that is being sought by the researcher, veterinarian, medical doctor or other clinician. Dosages particularly fall within a range of circulating concentrations that includes the ED₅₀ with little or no toxicity. Dosages may vary within this range depending upon the dosage form employed and/or the route of administration utilized. the exact formulation, route of administration, dosage, and dosage interval should be chosen according to methods known in the art, in view of the specifics of a subject’s condition.

Dosage amount and interval may be adjusted individually to provide plasma levels of the active moiety that are sufficient to achieve the desired effects; i.e., the minimal effective concentration (MEC). the MEC will vary for each compound but can be estimated from, for example, in vitro data and animal experiments. Dosages necessary to achieve the MEC will depend on individual characteristics and route of administration. In cases of local administration or selective uptake, the effective local concentration of the drug may not be related to plasma concentration.

The amount of agent or composition administered may be dependent on a variety of factors, including the sex, age, and weight of the subject being treated, the severity of the affliction, the manner of administration, and the judgment of the prescribing physician.

A unit dose form is a term that is generally understood by the skilled artisan. A unit dose forms is a pharmaceutical drug product that is marketed for a specific use. The drug product includes the active ingredient(s) and any inactive components, most often in the form of pharmaceutically acceptable carriers or excipients. It is understood that multiple unit dose forms are distinct drug products.

In a further embodiment, the invention is directed to unit dosage forms comprising MMB dihydrochloride monohydrate. In some embodiments, the unit dosage form comprises MMB, or a pharmaceutically acceptable salt thereof, in amount equivalent to from about 10 mg to about 1000 mg, about 10 mg to about 800 mg, about 10 mg to about 700 mg, about 10 mg to about 500 mg, about 10 mg to about 400 mg, about 10 mg to about 300 mg, about 10 mg to about 250 mg, about 10 mg to about 200 mg, about 10 mg to about 150 mg, about 10 mg to about 100 mg, about 10 mg to about 50 mg, about 50 mg to about 1000 mg, about 50 mg to about 800 mg, about 50 mg to about 700 mg, about 50 mg to about 500 mg, about 50 mg to about 400 mg, about 50 mg to about 300 mg, about 50 mg to about 250 mg, about 50 mg to about 200 mg, about 50 mg to about 150 mg, about 50 mg to about 100 mg, about 100 mg to about 1000 mgs, about 100 mg to about 800 mg, about 100 mg to about 700 mg, about 100 mg to about 500 mg, about 100 mg to about 400 mg, about 100 mg to about 300 mg, about 100 mg to about 250 mg, about 100 mg to about 200 mg, about 150 mg to about 300 mg, about 150 mg to about 250 mg, about 150 mg to about 200 mg, about 200 mg to about 300 mg, about 200 mg to about 250 mg, or about 200 mg to about 300 mg. In some cases, the amount is determined on the basis of MMB free base present.

### Pharmaceutical Compositions

Methods for treatment of joint inflammation (e.g., rheumatoid arthritis) are described herein. Said methods of the disclosure include administering a therapeutically effective amount of MMB. The MMB can be formulated in pharmaceutical compositions. These compositions may comprise, in addition to the active compound(s), a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer’s Injection, Lactated Ringer’s Injection. Preservatives, stabilizers, buffers, antioxidants and/or other additives can be included, as required.

The present technology is not limited to any particular composition or pharmaceutical carrier, as such may vary. In general, compounds of the present technology will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of the present technology is inhalation.

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. there are several types of pharmaceutical inhalation devices-nebulizer inhalers, metered dose inhalers (MDI) and dry powder inhalers (DPI). Nebulizer devices produce a stream of high velocity air that causes therapeutic agents (which are formulated in a liquid form) to spray as a mist that is carried into the subject’s respiratory tract. MDI’s typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the subject’s inspiratory air-stream during breathing by the device. In order to achieve a free flowing powder, therapeutic agent is formulated with an excipient such as lactose. A measured amount of therapeutic agent is stored in a capsule form and is dispensed with each actuation.

Pharmaceutical dosage forms of a compound of the present technology may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the compositions of the present technology can include one or more physiologically acceptable inactive ingredients that facilitate processing of active molecules into preparations for pharmaceutical use.

Pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of the present technology in combination with at least one pharmaceutically acceptable excipient. Acceptable excipients are non-toxic, aid administration, and do not adversely affect therapeutic benefit of the claimed compounds. Such excipient may be any solid, liquid, semisolid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including oils of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of the present technology in aerosol form. Inert gases suitable for this purpose are nitrogen, carbon dioxide, etc. Other suitable pharmaceutical excipients and their formulations are described in Remington’s Pharmaceutical Sciences, edited by E. W. Martin (Mack Publishing Company, 18th ed., 1990).

The present compositions may, if desired, be presented in a pack or dispenser device containing one or more unit dosage forms containing the active ingredient. Such a pack or device may, for example, comprise metal or plastic foil, such as a blister pack, or glass, and rubber stoppers such as in vials. the pack or dispenser device may be accompanied by instructions for administration. Compositions comprising a compound of the present technology formulated in a compatible pharmaceutical carrier may also be prepared, placed in an appropriate container, and labeled for treatment of an indicated condition.

The amount of the compound in a formulation can vary within the full range employed by those skilled in the art. Typically, the formulation will contain, on a weight percent (wt %) basis, from about 0.01-99.99 wt% of a compound of the present technology based on the total formulation, with the balance being one or more suitable pharmaceutical excipients. Preferably, the compound is present at a level of about 1-80 wt%.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature.

### Methods

### Animal Housing

Lewis rats and DBA/1 mice are purchased from Charles River. Lewis rats are immunized up to 3 days after arrival. Mice are purchased at the weaning age and allowed to acclimate for 3 weeks prior to commencing the experiment. All animals are housed with the 12:12 hour light: dark cycle, with the standard animal chow (SNIFF, 166 mg/kg iron) provided ad libitum. The health state and welfare of the animals is monitored at least once daily.

### PG-PS Rat Arthritis Model

Six-week old female Lewis rats are immunized with 15 mg/kg Streptococcal Peptidoglycan-Polysaccharide (PG-PS) administered intraperitoneally (IP). Treatments with anti-inflammatory drugs of interest are commenced two weeks later and continue for 7 days (short-term) to 21 days (long-term). Disease progression is monitored as described below (see Joint Pathology, Clinical Scoring and Bio-Imaging). Blood samples are collected once weekly and processed as described below. At the conclusion of experiments, blood, serum, and organ samples are collected and processed as described herein.

### Collagen Antibody-Induced Arthritis (CAIA) Model

Six-week old male mice are immunized with chicken type II collagen (200 µg/dose/animal) in complete Freund’s adjuvant (CFA, 200 µL emulsion per mouse) administered intradermally at the tail base twice in a span of 14 days. The treatments with anti-inflammatory drugs of interest will be commenced on day 28 after the first immunization and continued for either 7 additional days (short-term) or 21 additional days (long-term). Development of symptoms are monitored intravitally by caliper measurements of the hind limb joint diameters and determination of the clinical score based on the animal’s joint swelling. At the conclusion of experiments, blood, serum, and organ samples are collected and processed as described herein.

### Tissue Processing and Cell Isolation

Spleen and lymph node cells are isolated by mechanical pressing of the organ through a 100 µm cell strainer (Corning) in PBS. For isolation of rat synovial infiltrates, hind limbs are freed of skin, bones, and major tendons. The remaining tissue is minced and digested with 0.16 U Liberase TM, 10 µg/mL DNase I, and 60 U hyaluronidase I in RPMI at 37° C. for 1 hour. Blood is collected into heparinized tubes and used directly for blood count determination. For immunophenotyping of blood leukocytes, blood samples are depleted from red blood cells by lysis with ACK buffer (150 mM NH₄Cl, 10 mM KHCO₃, 0.1 mM EDTA).

### Hematological and Iron Parameter Determination and Histological Staining

Blood counts are measured with a Scil ABC Vet Counter (Horiba Medica).

Transferrin and hepcidin concentrations are assessed with commercial ELISA kits. Serum/plasma and organ non-heme iron concentration are measured with commercial kits from BioAssay Systems.

Serum and tissue iron content are determined using a commercially available kit (QuantiChrom). Iron deposits in spleen and liver are visualized using Pearls’s Prussian Blue staining in 3-5 µm thick sections of formalin-fixed, paraffin embedded material with Iron Stain Kit (Sigma).

### Flow Cytometry

Flow cytometry staining is performed as described in Parajuli et al., Int. J. Cancer 126:896-908, 2010, which is hereby incorporated by reference in its entirety. Measurements are performed with a Cytoflex S device (Beckman Coulter) and FACS Symphony (Beckton Dickinson), enabling detection of 13 and 52 fluorescence parameters, respectively, as well as automated absolute cell count determination.

For intracellular cytokine/transcription factor staining, rat and murine T cells are re-stimulated for 4 hours with phorbol 12,13-dibutyrate (PDBu) and ionomycin (50 ng/mL and 500 ng/mL, respectively) in the presence of Golgi Stop (BD Bioscience). In sum, five multicolor antibody panels per species (rat, mouse) are established (blood leukocytes, organ leukocytes, intracellular staining for Th cell subtypes, blood reticulocytes, bone marrow erythropoiesis). Data analysis is performed using FlowJo Software.

### Immunofluorescence Microscopy

Immunofluorescence (IF) microscopy is used as a primary technique for visualization of mouse joint infiltration with T cells and granulocytes. More precise immunophenotyping with flow cytometry of murine synovial tissue is not practical due to low cell yield after enzymatic tissue digestion.

IF microscopy is performed with formalin-fixed, paraffin-embedded mouse ankle joint material as described in Tymoszuk et al., Eur. J. Immunol. 44:2247-2262, 2014 and Tymoszuk et al., BMC Cancer 14:257, 2014, each of which is hereby incorporated by reference in its entirety. In brief, 3-5 µm sample sections are deparaffinized, rehydrated and subjected to antigen retrieval in citrate buffer, pH 6.0 at 95° C. Staining is performed with anti-CD3 (clone 17A2), anti-CD4 (GK1.5), anti-IL-17A (TC11-18H10), anti-Ly6G (1A8), anti-Ly6C (HK1.4) and F4/80 (BM8) antibodies and appropriate secondary reagents. Samples are photographed with an Axioscope (Zeiss) fluorescence microscope.

### Cytokine Determination

RNA and protein isolation, qRT PCR and Western Blotting with organ samples (liver, spleen, synovial tissue) will be performed as described in Asshoff et al., Blood 129: 1823-1830, 2017 which is hereby incorporated by reference in its entirety. In brief, qRT PCR are utilized to determine transcript levels for selected iron turnover proteins (such as Fpn1/Slc40a1, TfR1 and hepcidin), transcriptional targets of the JAK/STAT (Irf1, Socs1, Socs3, Stat1, Stat3) and AVCR1/SMAD pathways (Smad1/2/3/4, Fos, Pdgfb, Duspl) as well as selected cytokine, inflammatory and Th subset hallmark genes (Il1b, Il2, Il4, IL5, Il6, Il8, Il10, IL12a, IL12b, Il17a, Il21, Il22, Tgfb1, Tnf, CRP, iNOS, Arg1, Rankl, MMP1, MMP4, MMP6, MMP9, BMP4). Western Blotting is used to assess protein levels of iron turnover proteins (FPN1, ferritin, TfR1), activity of the JAK/STAT (phosphorylated and total STAT1 and STAT3) and AVCR1/SMAD pathways (phosphorylated and total SMAD1/2/3, total SMAD4).

Serum and recall culture supernatant cytokine levels are determined with ProcartaPlex cytokine panels (Th cytokine subset panel for the mouse: EPX170-26087-901, rat: EPX140-30120-901, both from Thermo Fisher) and the multiplex detection pipeline available at the Medical University Innsbruck.

### Th17 Recall Assays

Splenocytes and lymph node cells isolated from control and drug-treated CAIA animals are cultured in 96 well plates with 50 µg/mL chicken collagen in RPMI1640 medium supplemented with penicillin, streptomycin, L-glutamine, pyruvate, and betamercaptoethanol. Cytokine concentration in culture supernatant is measured by cytokine multiplex two days after culture onset.

### Joint Pathology, Clinical Scoring and Bio-Imaging

Regular monitoring of arthritis progression in all animal models is accomplished by measuring hind limb ankle joint diameters with a Vernier’s caliper (volume = D × d²Π/6, D and d are ankle diameters measured in perpendicular axes, D > d). In murine arthritis models, animals are additionally clinically scored with a system assessing swelling and mobility of each animal’s limb, as described in Inglis et al., Arthritis Res. Ther. 8:R113, 2007, which is hereby incorporated by reference in its entirety. Each paw of an experimental animal is assessed separately as follows: 0 - normal, 1 - slight swelling or erythrema, 2 - pronounced swelling, 3 - joint rigidity. The paw scores for each animal are summed giving a maximum of 12 points per mouse. Scoring is performed by a rater blinded to the group assignment.

Two non-invasive bio-imaging tools — micro-CT and MRI (Magnetic Resonance Imaging) — are used in combination with image data analysis to trace disease progression in rodent models of arthritis.

At necropsy, selected representative animal limbs are freed of coat and skin and fixed in formalin and embedded in paraffin. Joint pathology and parameters such as leukocyte infiltration, tendon, bone and cartilage damage are investigated in 3 - 5 µm thick tissue slides stained with HE (mouse and rat) and IF microscopy (mouse only).

### Disease Monitoring and Intravital Blood Sample Workup

Joint volume is measured weekly and micro-CT scans are performed with selected representative animals from each treatment group. In the CAIA model, mice are also clinically scored in weekly intervals. For intravitally collected blood samples, the following parameters are ascertained: blood counts (Hb, WBC, RBC, MCV and reticulocytes), plasma TF-Sat, iron and hepcidin concentrations, blood leukocyte immunophenotyping by flow cytometry, and plasma cytokine concentration by cytokine complex. Additional measurements can be performed on the frozen plasma samples.

### Example 1: Assessing Momelotinib (MMB) Treatment of Arthritis in a Rat Model

A rat model of chronic arthritis (PG-PS induced) was used to assess the efficacy of the dual JAK1/2 and ACVR1 inhibitor momelotinib (MMB) in vivo. Two weeks following arthritis induction by immunization with PG-PS, rats were treated with daily oral doses of MMB (5, 10, and 25 mg/kg) or vehicle for 3 weeks.

Consecutive assessment of arthritis was performed by joint thickness measurements and paw scoring. Following 3 weeks of treatment, synovial immune cell infiltration and T cell subset differentiation were quantified by multi-color flow cytometry. Cytokine gene expression was profiled by Real-Time PCR. Anemia was assessed by determination of blood hemoglobin and serum, spleen and liver iron levels.

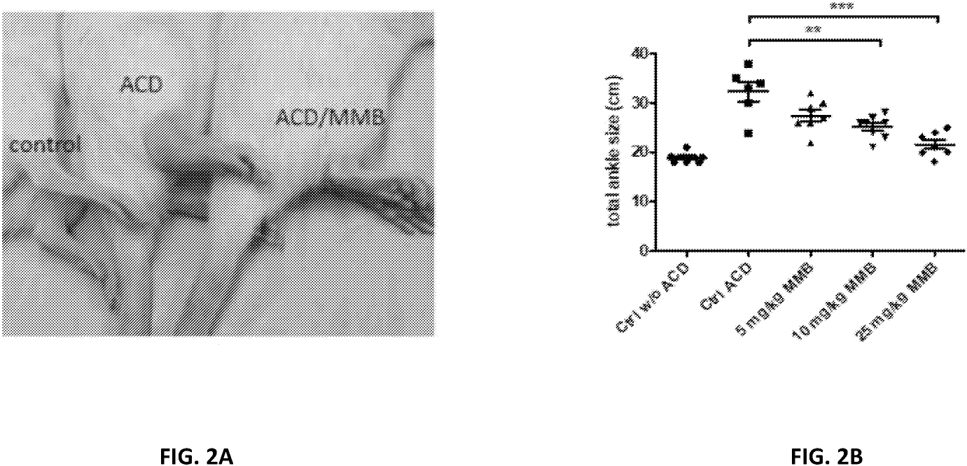

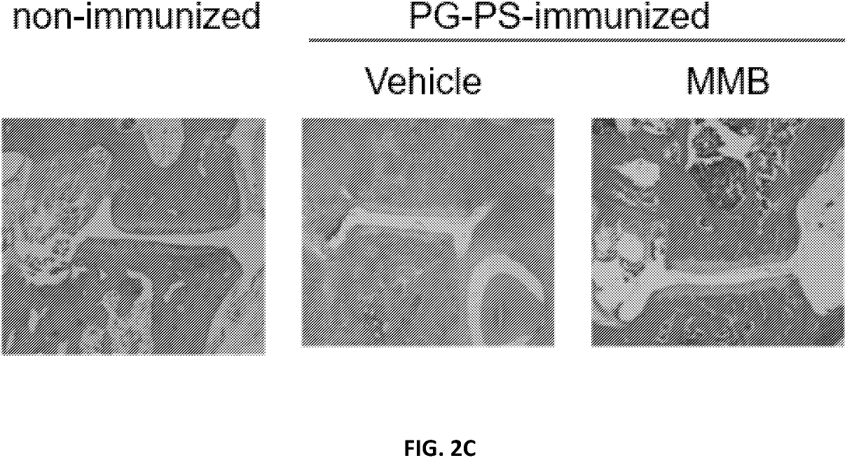

A significant reduction in the mean sum diameter (± SEM [n = 8 pergroup]) of hind limb joints was observed with daily administration of MMB at 10 and 25 mg/kg as measured by caliper on the last day of treatment (FIG. 2A and FIG. 2B). Statistical significance was determined with one-way ANOVA with Bonferroni post-hoc tests (** p < 0.01, *** p < 0.001). FIG. 2C shows representative photos of H&E-stained sections of hind limb ankle joins from non-immunized rats and PG-PS-immunized rats treated with vehicle or MMB (10 mg/kg). The results demonstrate that the dual JAK1/2 and ACVR1 inhibitor MMB substantially inhibits swelling and cartilage destruction in arthritic hind limb joints.

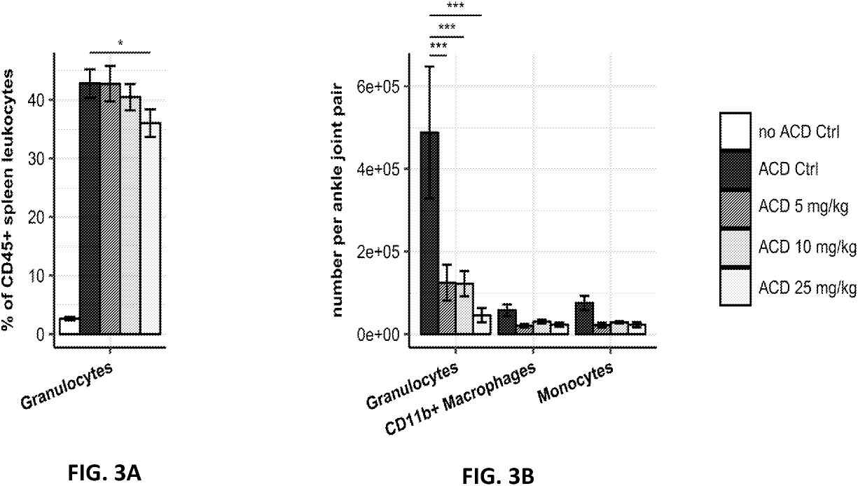

Spleen and synovial tissue samples were taken from all animals and evaluated by flow cytometry in order to assess the myeloid cell population in each tissue in the presence and absence of MMB treatment. PG-PS-immunized rats treated with 25 mg/kg MMB for 3 weeks showed significant reduction in splenic granulocytes (CD45⁺, CD11b/c⁺, Granulocyte⁺) (FIG. 3A). MMB treatment also significantly reduced granulocytes (CD45⁺, CD11b/c⁺, Granulocyte⁺), inflammatory CD11b/c⁺ macrophages (CD45⁺ Granulocyte⁻ CD11b/c⁺ Macrophage⁺), and monocytes (CD45⁺ Granulocyte⁻ CD11b/c⁺ Macrophage⁻) in synovial tissue isolated from hind limb ankle joint pairs of rats immunized with PG-PS (FIG. 3B). Splenic granulocytes (percentage of total splenic CD45⁺ leukocytes) and synovial myeloid cells (total counts per ankle joint pair) were determined by flow cytometry and are presented as mean ± SEM (n = 6-10 per group). Statistical significance was determined with one-way ANOVA with Bonferroni post-hoc tests (* p < 0.05, *** p < 0.001). These results suggest that the anti-arthritic activity following MMB treatment is due to a significant reduction of inflammatory myeloid cell infiltration into the synovia.

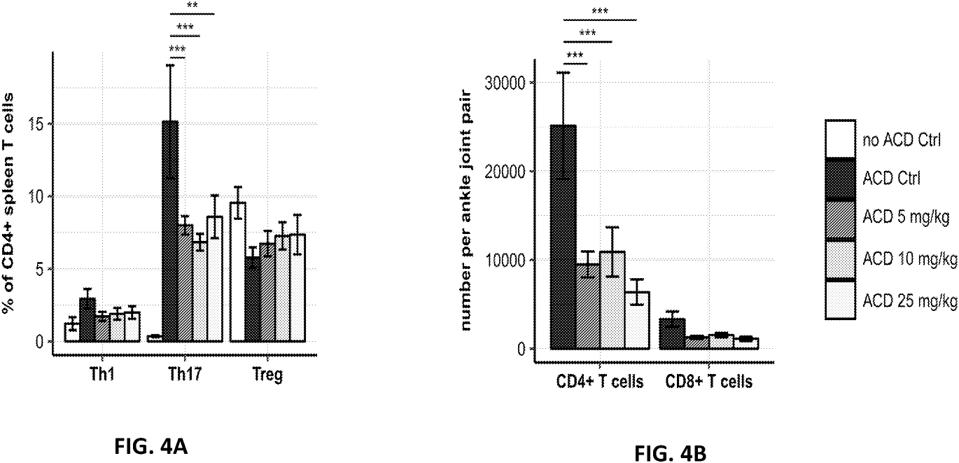

Samples from spleen tissue were also used to assess levels of Th1, Th17, and Treg cells in the spleen in non-immunized rats, as well as rats immunized with PG-PS and treated daily with vehicle or various concentrations of MMB for 3 weeks. Results show that MMB treatment resulted in significant reduction in splenic Th17 cells (CD45⁺, CD3⁺, CD8⁻, CD4⁺, FOXP3⁻, IFN-gamma⁻, IL17A⁺) and reduced splenic Th1 cells (CD45⁺, CD3⁺, CD8⁻, CD4⁺, FOXP3⁻, IFN-gamma⁺, IL17A⁻) (FIG. 4A). Splenic Treg cells (CD45⁺, CD3⁺, CD8⁻, CD4⁺, FOXP3⁺) were not significantly altered following MMB treatment.

Samples of synovial tissue from rat hind limb ankle joints were used to assess levels of CD4⁺ T cells and CD8⁺ T cells in animals treated with vehicle or various concentrations of MMB. MMB treatment resulted in significant reductions in synovial CD4⁺ and CD8⁺ T cells (FIG. 4B).

Splenic T cell percentages (of CD4⁺ T cells) (FIG. 4A) and absolute T cell counts per hind limb ankle joint (FIG. 4B) were determined by flow cytometry. Reduction in CD4⁺ T cells in the arthritic ankle synovia was observed following treatment with even the lowest dose of MMB tested (5 mg/kg) (FIG. 4B). Data are presented as mean ± SEM (n = 6-10 per group). Statistical significance was determined with one-way ANOVA with Bonferroni post-hoc tests (^{∗} p < 0.05, ^{∗∗∗} p < 0.001). The data suggest that the anti-arthritic activity of MMB treatment is at least in part through inhibition of Th17 cell differentiation and T cell recruitment into synovial tissue.

MMB treatment reduced inflammatory granulocyte and macrophage infiltration in synovial tissue by more than 60% at all tested doses as compared to vehicle treatment in PG-PS animals. MMB treatment effectively decreased arthritogenic Th17 cell differentiation and overall CD4⁺ T cells in the synovia beginning at the lowest tested dose (5 mg/kg) and coincided with complete remission of joint swelling at 25 mg/kg.

### Example 2: Evaluting Efficacy of Momelotinib (MMB) for Treating Collagen Antibody-Induced Arthritis (CAIA) in DBA/1 Mice

In order to evaluate efficacy of MMB treatment in a second arthritic animal model, arthritis was induced in DBA/1 mice by intravenous injection of collagen antibody cocktail on day 0 followed by intraperitoneal injection of lipopolysaccharide (LPS) on day 3. Beginning on day 0, mice were treated orally for the entirety of the study either once daily vehicle, MMB (20 or 50 mg/kg) or twice daily with MMB (30 mg/kg). Comparator controls dexamethasone (1 mg/kg) or the TNF-α inhibitor, etanercept (10 mg/kg) were administered daily by intraperitoneal injection beginning on day 0. A schematic outlining the experimental strategy is shown in FIG. 5.

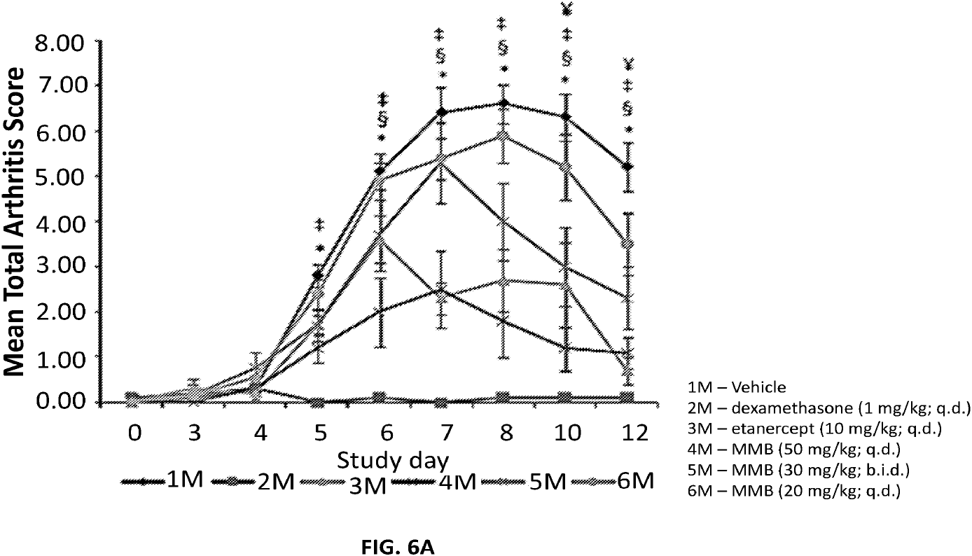

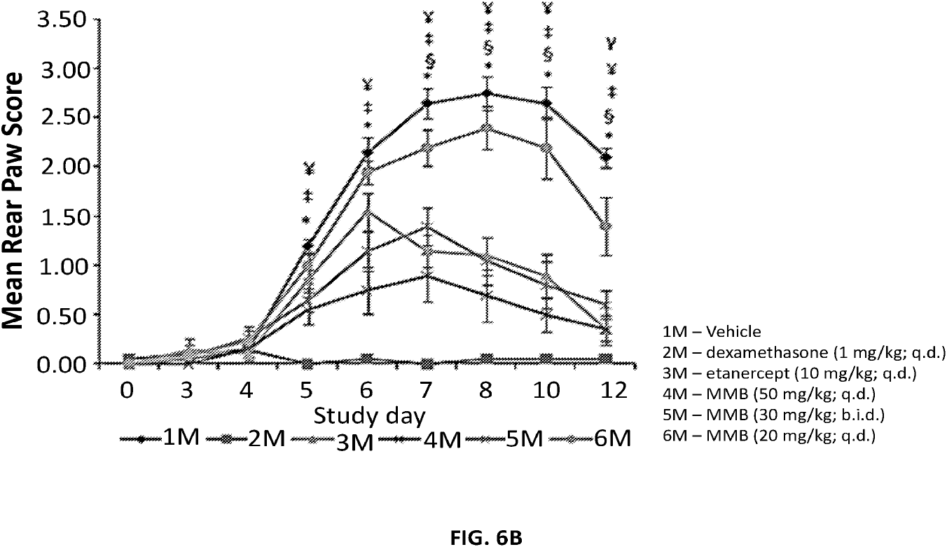

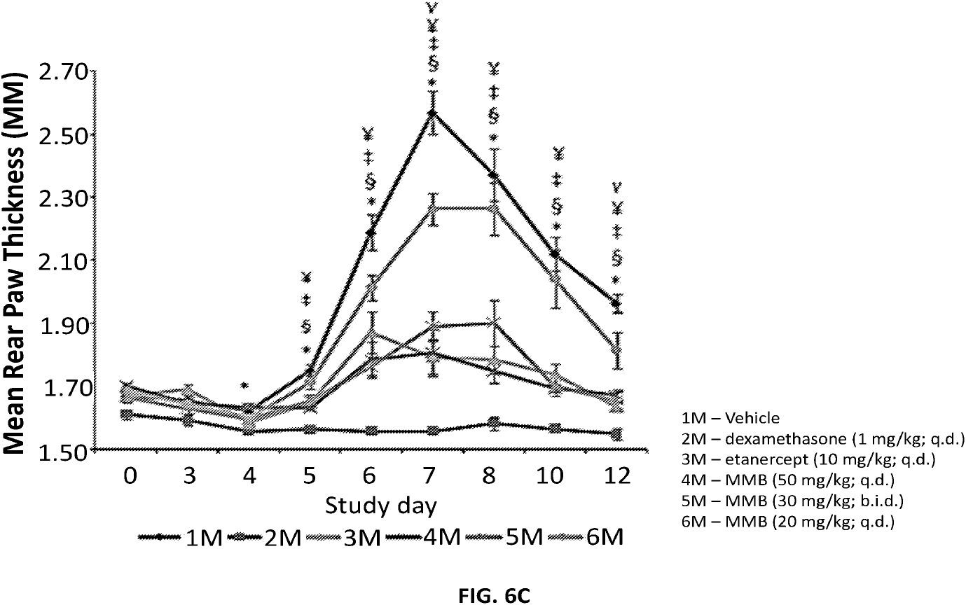

Beginning on day 5, a significant reduction in the mean total (FIG. 6A) and rear paw arthritis scores (FIG. 6B) and mean rear paw thickness (FIG. 6C) were observed following daily administration of MMB (50 mg/kg daily or 30 mg/kg twice daily) or etanercept compared to vehicle. MMB treatment (20 mg/kg daily) also resulted in reduction of mean rear paw thickness on days 7 and 12 compared to vehicle (FIG. 6C). Dexamethasone treatment resulted in reduction in all measurements from day 4 to day 12 (FIGS. 6A-6C). These results confirm the anti-arthritic activity of MMB with significant and sustained reductions in arthritis scoring, which demonstrated non-inferiority versus the TNF-α inhibitor, etanercept, in the mouse collagen antibody-induced arthritis (CAIA) model.

The anti-arthritic activity of MMB was confirmed with significant and sustained reductions in arthritis scoring, which demonstrated non-inferiority versus the TNF-α inhibitor, etanercept, in the CAIA model. Consistent with its inhibitory activity on the ACVR1-hepcidin axis, MMB reduced circulating hepcidin levels and mobilized systemic iron, resulting in substantial improvement of the RA-associated anemia in rats.

### Example 3: Assessing Momelotinib (MMB) Treatment of Rheumatoid Arthritis in a Rat Model

### Rat rheumatoid Arthritis (RA) Model

Female Lewis rats were immunized with 15 mg/kg Streptococcal Peptidoglycan-Polysaccharide (PG-PS) administered intraperitoneally (IP) to generate a rat model of rheumatoid arthritis. Rats were used to assess the efficacy of the dual JAK1/2 and ACVR1 inhibitor momelotinib (MMB) in vivo. Two weeks following rheumatoid arthritis induction by immunization with PG-PS, rats were treated with daily oral doses of MMB (5, 10, 25 mg/kg) or vehicle for 3 or 21 days.

Statistical significance was determined with one-way ANOVA for RA animals with Bonferroni post-hoc tests (N=5-10 per group, * p < 0.05, ** p < 0.01).

Cytokine gene expression was profiled by Real-Time PCR. For immunophenotyping of blood leukocytes and granulocyte counts were obtained. Anemia was assessed by determination of blood hemoglobin and serum, spleen and liver iron levels.

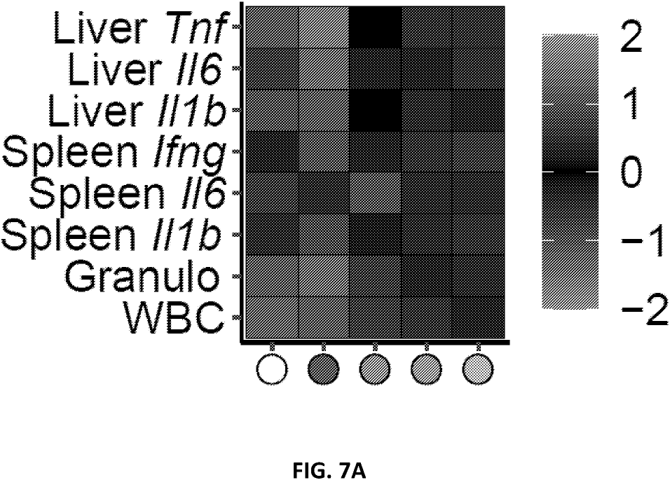

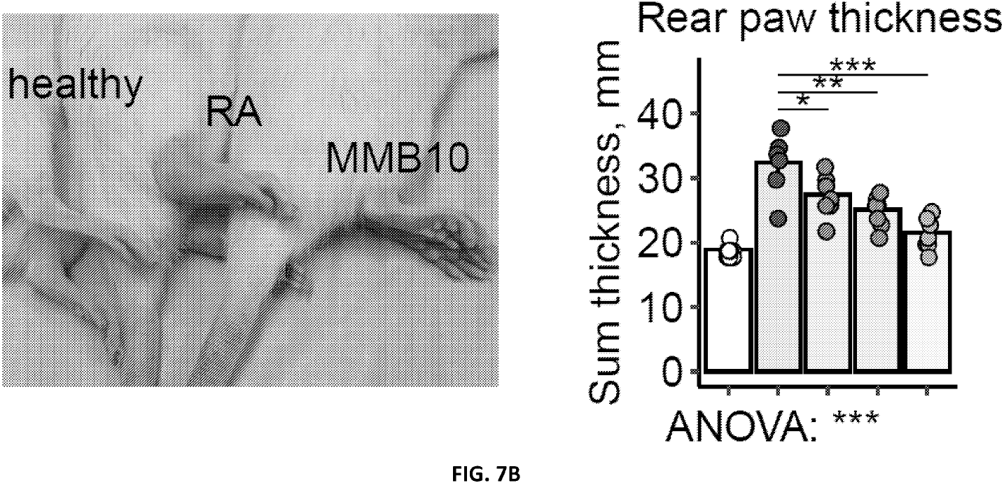

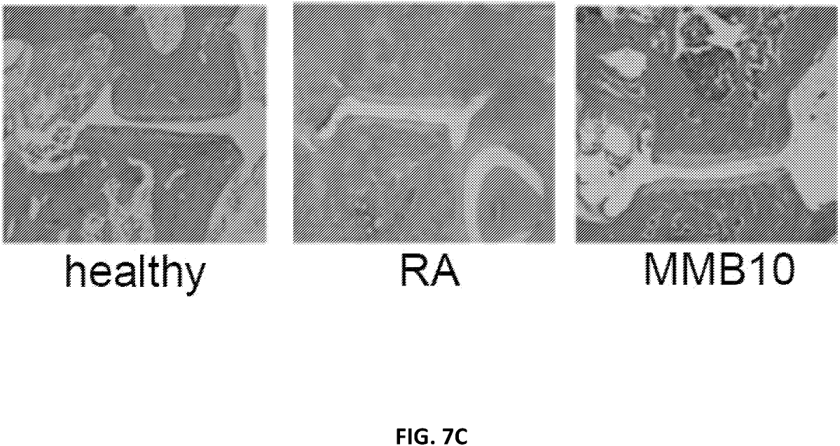

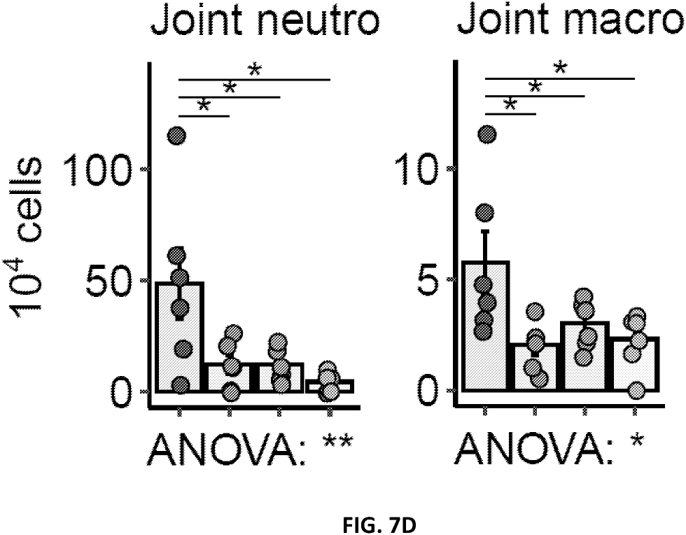

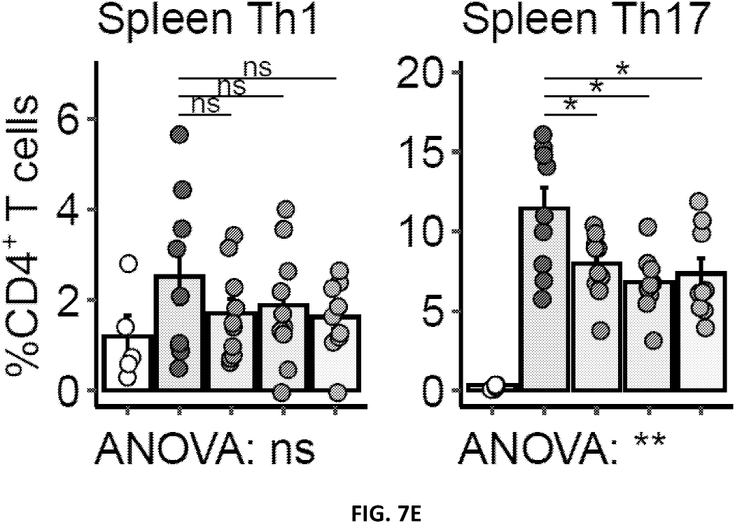

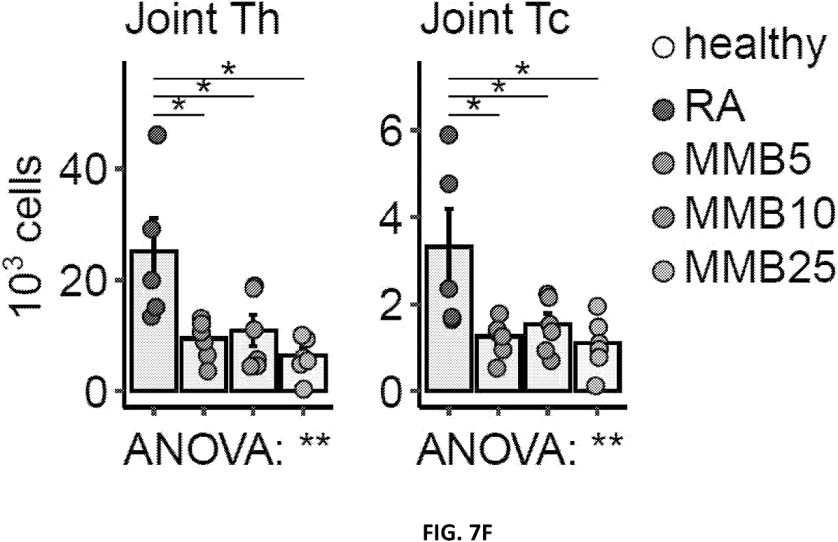

Following 3 days of treatment, MMB treatment significantly decreases spleen and liver cytokine expression, blood leukocyte and granulocyte counts (FIG. 7A). Data were normalized, group-wise means presented as a heat map.

Following 21 days of treatment, MMB reduces hind limb joint swelling. FIG. 7B shows representative photos and sum hind limb thickness of healthy, rheumatoid arthritis and MMB 10 (10 mg/kg) treated rats.

Following 21 days of treatment, MMB therapy alleviates cartilage damage. FIG. 7C shows representative H&E-stained hind limb joint sections.

Following 21 days of treatment, MMB reduces infiltration of neutrophils (CD45⁺ CD11b/c⁺ Granulocyte⁺), and macrophages (CD45⁺ Granulocyte- CD11b/c⁺ Macrophage⁺) in hind limb synovia of animals. FIG. 7D presented flow cytometry (FC) data of joint neutrophils and joint macrophages in rats receiving MMB treatment.

FIG. 7E presents flow cytometry data showing MMB inhibits differentiation of splenic Th17 cells (CD45⁺, CD3⁺, CD4⁺, FOXP3⁻, IFN-γ⁻, IL17A⁺) after 21 days of treatment without affecting Th1 (CD45⁺, CD3⁺, CD4⁺, FOXP3⁻, IFN-γ⁺, IL17A⁻) and regulatory Treg cells (CD45⁺, CD3⁺, CD8⁻, CD4⁺, FOXP3⁺) of RA rats compared with healthy rats.

FIG. 7F presents flow cytometry data showing MMB significantly reduces CD4⁺ and CD8⁺ T cell infiltration in hind-limb synovial tissue after 21 days of treatment.

Following 21 days of treatment, MMB decreases hepcidin and corrects anemia in the PG-PS rat RA when compared to healthy and RA rats (FIG. 8).

Janus kinases (JAK) orchestrate inflammation, immunity and erythropoiesis. Several JAK inhibitors (JAKi) are approved for RA treatment. Exacerbation of RA-associated anemia is a common side effect of JAKi. MMB, a JAK1/2 and activin receptor 1 (ACVR1) inhibitor, ameliorates anemia in the PG-PS rat RA model and myelofibrosis patients.

Here, MMB shows significant anti-RA activity in the rat PG-PS arthritis model. MMB ameliorates systemic inflammation, infiltration of immune cells into synovial tissue and inhibits differentiation of arthritogenic Th17 cells in the rat PG-PS model. In addition to the anti-arthritic activity, MMB treatment reduces hepcidin, improves iron availability and corrects RA-associated anemia. Further, MMB reduces inflammation-driven hepcidin production, improves iron availability and erythropoiesis via ACVR1 inhibition.

### Example 4: Clinical Activity and Non-inferiority of Momelotinib (MMB) in the CAIA murine Rheumatoid Arthritis

### Murine Rheumatoid Arthritis (RA) Model

BALB/C mice were immunized with 2 mg anti-collagen II antibodies administered intraperitoneally (IP) on day 0 followed by intraperitoneal injection of 100 µg of lipopolysaccharide (LPS) on day 3. Beginning at RA induction, mice were treated orally for the entirety of the study either once daily vehicle, MMB (50 mg/kg), twice daily with MMB (30 mg/kg), or daily intraperitoneally, etanercept (ETA) (10 mg/kg).

Consecutive assessment of rheumatoid arthritis was performed by animal and rear paw scoring, and rear paw thickness measurements.

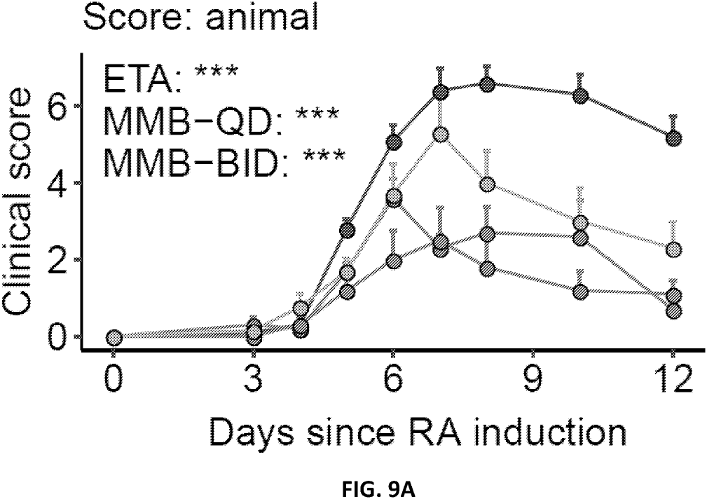

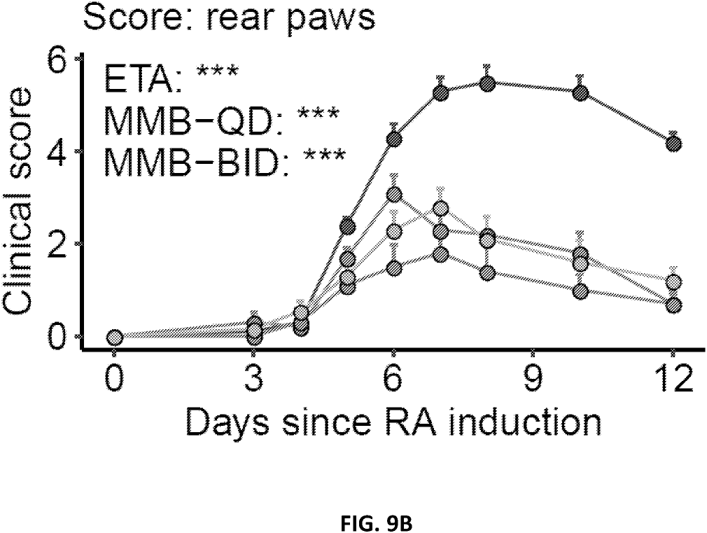

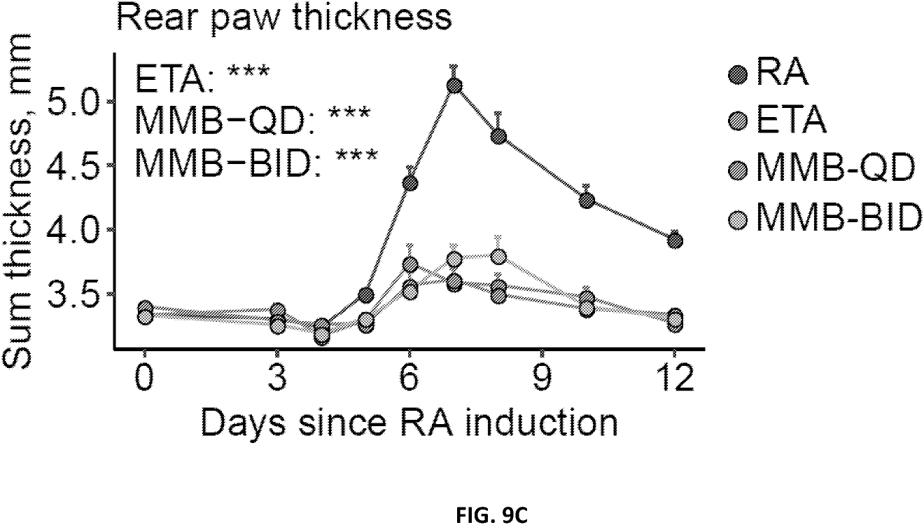

Beginning on day 6, a significant reduction in the mean total animal (FIG. 9A) and rear paw rheumatoid arthritis scores (FIG. 9B) and mean rear paw thickness (FIG. 9C) were observed following daily administration of MMB (50 mg/kg daily or 30 mg/kg twice daily) or etanercept compared to vehicle. These results support the anti-arthritic activity of MMB with significant and sustained reductions in rheumatoid arthritis scoring, which demonstrated non-inferiority versus the TNF-α inhibitor, etanercept, in the mouse collagen antibody-induced arthritis (CAIA) model. Statistical significance was determined with repeated-measure ANOVA for RA-therapy comparison (N=10-13 per group, *** p < 0.001).

MMB displays significant anti-arthritic activity in the murine CAIA RA model and proves at least equally effective as etanercept. Collectively, these data strongly suggest that MMB treatment can address local and systemic inflammation and consequent anemia in rodent models of arthritis.

### 6. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

What is claimed is:

1. A method of treating joint inflammation, the method comprising:
administering to a subject in need thereof a therapeutically effective amount of momelotinib (MMB) or a pharmaceutically acceptable salt thereof.

2. The method of claim 1, wherein the subject has arthritis.

3. The method of claim 2, wherein the subject has rheumatoid arthritis.

4. The method of any of the preceding claims, wherein treating joint inflammation comprises at least ameliorating one or more symptoms of arthritis.

5. The method of claim 4, wherein at least ameliorating one or more symptoms of arthritis comprises a reduction in joint volume.

6. The method of claim 5, wherein the reduction in joint volume is at least 2% compared to the joint volume prior to administering the therapeutically effective amount of MMB.

7. The method of claim 6, wherein the reduction in joint volume is at least 5% compared to the joint volume prior to administering the therapeutically effective amount of MMB.

8. The method of any of the preceding claims, wherein treating joint inflammation comprises ameliorating swelling of affected tissue, as measured by a reduction in diameter of an inflamed joint.

9. The method of claim 8, wherein the reduction in diameter of the inflamed joint is at least 2% compared to diameter of the inflamed joint prior to administering the therapeutically effective amount of MMB.

10. The method of any of the preceding claims, wherein treating joint inflammation comprises reducing blood neutrophil count in the subject following administering the therapeutically effective amount of MMB.

11. The method of any of the preceding claims, wherein treating joint inflammation comprises reducing myeloid cell infiltration in spleen and/or synovial tissue.

12. The method of claim 11, comprising reducing in synovial tissue at least one of granulocytes, macrophages, monocytes and neutrophils.

13. The method of claim 12, wherein the inflammatory macrophages are CD11b⁺.

14. The method of claim 12, comprising reducing in synovial tissue the amount of at least one of granulocytes or macrophages compared to the amount of at least one of granulocytes or macrophages in synovial tissue from the subject prior to administering MMB.

15. The method of claim 14, wherein the amount of at least one of granulocytes or macrophages is reduced by at least 10%.

16. The method of claim 15, wherein the amount of at least one of granulocytes or macrophages is reduced by at least 15%.

17. The method of claim 16, wherein the amount of at least one of granulocytes or macrophages is reduced by at least 20%.

18. The method of claim 17, wherein the amount of at least one of granulocytes or macrophages is reduced by at least 30%.

19. The method of claim 18, wherein the amount of at least one of granulocytes or macrophages is reduced by at least 40%.

20. The method of claim 19, wherein the amount of at least one of granulocytes or macrophages is reduced by at least 50%.

21. The method of claim 20, wherein the amount of at least one of granulocytes or macrophages is reduced by at least 60%.

22. The method of any of the preceding claims, wherein administering the therapeutically effective amount of MMB or a pharmaceutically acceptable salt thereof results in reduction in the number of IL-17A-producing helper T lymphocytes (Th17 cells) in the spleen of the subject compared to the number of Th17 cells in the spleen of the subject prior to administering MMB or a pharmaceutically acceptable salt thereof.

23. The method of any of the preceding claims, wherein administering the therapeutically effective amount of MMB or a pharmaceutically acceptable salt thereof results in reduction in the number of CD4⁺ and/or CD8⁺ T cells in synovial tissue of an inflamed joint of the subject compared to the number CD4⁺ and/or CD8⁺ T cells in synovial tissue of the inflamed joint prior to administering MMB or a pharmaceutically acceptable salt thereof.

24. The method of any of the preceding claims, wherein the MMB or a pharmaceutically acceptable salt thereof is administered orally.

25. The method of any of the preceding claims, wherein the MMB or a pharmaceutically acceptable salt thereof is administered daily.

26. The method of any one of claims 1-24, wherein the MMB or a pharmaceutically acceptable salt thereof is administered weekly.

27. The method of any one of claims 1-24, wherein the MMB or a pharmaceutically acceptable salt thereof is administered intermittently.

28. The method of any one of claims 1-25, wherein the therapeutically effective amount of MMB or a pharmaceutically acceptable salt thereof is 25-500 mg/day.

29. The method of claim 28, wherein the therapeutically effective amount of MMB or a pharmaceutically acceptable salt thereof is selected from: 25 mg/day, 50 mg/day, 100 mg/day, 200 mg/day, 300 mg/day, 400 mg/day, and 500 mg/day.

30. The method of claim 29, wherein the therapeutically effective amount of MMB or a pharmaceutically acceptable salt thereof is 200 mg/day.

31. The method of claim 30, wherein the therapeutically effective amount of MMB or a pharmaceutically acceptable salt thereof is administered for a period of 1 week or more.

32. The method of claim 31, wherein the therapeutically effective amount of MMB or a pharmaceutically acceptable salt thereof is administered for a period of 2 weeks or more.

33. The method of claim 32, wherein the therapeutically effective amount of MMB or a pharmaceutically acceptable salt thereof is administered for a period of 3 weeks or more.

34. The method of claim 33, wherein the therapeutically effective amount of MMB or a pharmaceutically acceptable salt thereof is administered for a period of 1 month or more.

35. The method of any one of the preceding claims, wherein the subject is a mammal.

36. The method of any one of the preceding claims, wherein the subject is human.

35. A method of treating joint inflammation in a subject, the method comprising:
administering to a subject in need thereof a therapeutically effective amount of momelotinib (MMB) or a pharmaceutically acceptable salt thereof, and
administering to the subject one or more additional anti-inflammatory agents.

36. The method of claim 36, wherein the one or more additional anti-inflammatory agents are anti-arthritic agents.

37. A method of treating inflammation-associated anemia concurrently with treating joint inflammation in a subject, the method comprising:
administering to a subject in need thereof a therapeutically effective amount of momelotinib (MMB) or a pharmaceutically acceptable salt thereof.

38. The method of claim 37, wherein the subject has transferrin or hepcidin levels that are above the normal range and serum iron and transferrin saturation that are below the normal ranges.
